(19) United States
(12) Patent Application Publication
(10) Pub. No.: US 2009/0216260 A1
(43) Pub. Date: Aug. 27, 2009

(54) INTERLOCKING HANDLE

(76) Inventors: Alison M. SOUZA, Santa Clara, CA (US); Brian E. Martini, Aptos, CA (US); Gary B. Hulme, San Jose, CA (US); Jason A. Jegge, San Jose, CA (US)

Correspondence Address: SHAY GLENN LLP, 2755 CAMPUS DRIVE, SUITE 210, SAN MATEO, CA, 94403, US

(21) Appl. No.: 12/390,307

(22) Filed: Feb. 20, 2009

Related U.S. Application Data
(60) Provisional application No. 61/030,171, filed on Feb. 20, 2008.

(51) Int. Cl. A61B 17/34

(52) U.S. Cl. 606/185

(57) ABSTRACT

Described herein are interlocking surgical tools (e.g., trocars, cannula, etc.) that form a composite surgical device having a handle and a finger grip region that are separate, when the component portions are interlocked. In particular, these tools are configured so that the separate handle and finger grip regions are perpendicular to each other. This arrangement may provide benefits in manipulating these devices.

### CROSS-REFERENCE TO RELATED APPLICATIONS

This patent application claims priority to U.S. Provisional patent application Ser. No. 61/030,171, filed Feb. 20, 2008, and titled “IMPROVED INTERLOCKING HANDLE”.

This patent application may be related to U.S. application Ser. No. 12/024,938 (filed on Feb. 1, 2008), titled “SYSTEMS, DEVICES AND METHODS FOR STABILIZING BONE”).

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

Surgeons may use a variety of hand-held devices to gain access and treat interior body regions. These instruments may be used to penetrate tissue by the application of pushing forces, twisting forces, or both in combination. In addition, the instruments may provide channels for treatment or for addition and removal of material to and from the body. A single surgical procedure may require the physician to deploy more than one instrument, each a different shape, size and function. To meet these needs, instruments, including trocars and cannula, have been developed which may engage with each other.

For example, U.S. patent application Ser. No. 12/024,938, incorporated by reference in its entirety above, describes bone stabilization devices and methods for inserting them using one or more cannulas and one or more trocars. In performing such procedures, it would be extremely beneficial to have interlocking trocars and access cannula tools that can form a composite device that overcomes the difficulties just mentioned.

Composite tools and devices have been previously described, for example, in U.S. Pat. No. 6,575,919 to Reiley et al., which describes a hand-held composite instrument in which a first instrument engages with a second instrument to form a composite handle that may be manipulated. However such composite handles may not be appropriate for all uses. In particular these devices may not be well suited to the bone stabilization devices and methods method above. These prior art composite handles may be uncomfortable, as the connection between the two regions may pinch, and may be more difficult to manipulate, particularly when twisting or rotating either the composite device, or one or the component devices individually. Finally, the portion of the composite handle connected to one of the component devices (e.g., the trocar portion) must be positioned at the proximal end of the device, which may make it difficult to operate.

Described above are composite devices and methods of using them that may address these issues.

### SUMMARY OF THE INVENTION

Described herein are interlocking surgical tools (e.g., trocars, cannula, etc.) that form a composite surgical device having a handle and a finger grip region that are separate, when the component portions are interlocked. In particular, these tools are configured so that the separate handle and finger grip regions are perpendicular to each other. This arrangement may provide benefits in manipulating these devices. Although the tools forming the composite surgical devices described and illustrated here are primarily trocars and cannula, other surgical tools (e.g., needles, catheters, and the like) may also be used.

For example, described herein are interlocking trocar and access cannula tools configured to form a composite device. These tools may include a trocar and an access cannula. The access cannula may include an elongate cannula body; a finger grip surface extending from the cannula body, wherein the finger grip surface extends lateral to the cannula body from a position distal to the proximal end of the cannula body; an engagement region at the proximal end of the cannula body; and a stop at the proximal end region of the cannula body. The trocar may include: an elongate trocar body extending longitudinally; a handle at the proximal end of the trocar body, the handle having a gripping surface oriented parallel to the longitudinal axis of the trocar body; a mating surface at the distal portion of the handle configured to mate with the engagement region of the access cannula; and a stop mate within the engagement region. The trocar may be configured to slide within the elongate cannula body so that the engagement region on the access cannula mates with the mating surface on the trocar to form a composite device in which the finger grip surface of the access cannula is oriented perpendicular to the handle gripping surface of the trocar when the stop on the access cannula is mated with the stop mate on the trocar.

In any of these variations, the access cannula may include a pair of finger grips extending laterally from a portion the elongate body of the access cannula that is distal to the proximate end. The engagement region on the access cannula may include a threaded region that mates with the mating surface on the trocar.

In general, the device may be configured (e.g., via the stop and the stop mate) so that the finger grip on the access cannula is secured perpendicular (e.g., at a ninety degree angle) relative to the handle on the trocar. For example, the stop may comprise a projection extending from the proximal end of the access cannula, and the stop mate on the trocar may comprise a face configured to abut the projection extending from the proximal end of the access cannula when the trocar and access cannula are engaged and the finger grip surface is oriented at a right angle from the handle gripping surface of the trocar.

Although the example above is arranged so that the finger grip or grips are on the access cannula and the handle is on the trocar, this arrangement may be reversed. For example, an interlocking trocar and access cannula tool configured to form a composite device may include a trocar and an access cannula, where the trocar includes: a trocar handle at the proximal end of the trocar; a finger grip extending from a distal portion of the trocar handle, wherein the finger grip extends lateral to the trocar; a threaded engagement region at the proximal end of the trocar handle; and a stop extending from the proximal end region of the trocar handle. The access cannula may include a handle at the proximal end of the access cannula, the handle having a gripping surface; a threaded receiving region within the distal portion of the handle configured to mate with the threaded engagement region of the trocar; and a stop mate within the engagement region configured to engage the stop on the trocar handle. Similar to the variation described above, the access cannula may be configured to engage with the trocar to form a composite device so that the finger grip extending from the trocar is oriented approximately perpendicularly to the handle of the access cannula when the stop on the trocar is mated with the stop mate on the access cannula.

Furthermore, although the cannula is referred to as an access cannula, it may also simply be a cannula, or it may alternatively be referred to as an introducer cannula or an application cannula. In some variations of the device and methods described herein, the trocar may be replaced with a second cannula, or the access cannula may be replaced with a second trocar.

In some variations, the tool also includes a lock configured to secure the access cannula within the trocar when the two are engaged.

The finger grip surface of the access cannula may be configured to fit between a subject's fingers when the trocar is engaged with the access cannula to form the composite device and the subject is holding the composite device by the handle of the trocar. Furthermore, the finger grip surface of the access cannula may be configured so that it does not contact the handle of the trocar when the trocar is engaged with the access cannula to form the composite device.

Also described herein are interlocking trocar and access cannula tools configured to form a composite device. These tools may include an access cannula and a trocar. The access cannula may include: an access cannula handle at the proximal end of the access cannula; a finger grip extending from a distal portion of the access cannula handle, wherein the finger grip extends lateral to the access cannula; a threaded engagement region at the proximal end of the access cannula handle; and a stop extending from the proximal end region of the access cannula handle. The trocar may include: a handle at the proximal end of the trocar, the handle having a gripping surface; a threaded receiving region within the distal portion of the handle configured to mate with the threaded engagement region of the access cannula; and a stop mate within the engagement region configured to engage the stop on the access cannula handle. Similar to the variation described above, the access cannula may be configured to engage with the trocar to form a composite device so that the finger grip extending from the access cannula is oriented approximately perpendicularly to the handle of the trocar when the stop on the access cannula is mated with the stop mate on the trocar.

For example, in some variations, the access cannula includes a pair of finger grips extending laterally from the access cannula handle. The stop mate on the trocar may include a face configured to abut the stop extending from the proximal end of the access cannula when the trocar and access cannula are engaged and the finger grip is oriented at a right angle from the handle.

As mentioned above, the tools may include a lock configured to secure the trocar within the access cannula when the two are engaged.

The finger grip of the access cannula may be configured to fit between a subject's fingers when the trocar is engaged with the access cannula to form the composite device and the subject is holding the composite device by the handle of the trocar. Furthermore, the finger grip of the access cannula may be configured so that it does not contact the handle of the trocar when the trocar is engaged with the access cannula to form the composite device.

Also described herein are methods of manipulating an interlocking trocar and access cannula tool configured to form a composite device. For example, the method may include the steps of: inserting the distal end of a trocar into an access cannula, wherein the trocar includes a proximal handle and the access cannula includes at least on finger grip; connecting the proximal end of the access cannula with the distal portion of the handle on the trocar to form a composite device, so that a stop on the access cannula engages a stop mate on the trocar to secure the finger grip extending from the elongate body of the access cannula oriented at a right angle to the handle of the trocar; and manipulating the composite device while gripping the handle of the trocar.

The step of inserting the distal end of the trocar into the access cannula may comprise sliding the trocar within a channel through the access cannula. In some variations, the method may include the step of first inserting the access cannula into a subject's tissue.

The step of connecting the proximal end of the access cannula with the distal portion of the handle may include engaging an engagement region on the proximal end of the access cannula with a mating surface at the distal portion of the handle on the trocar.

The method may also include the step of locking the access cannula and the trocar together. In some variations, the method also comprising gripping the handle of the trocar so that the finger grip is positioned between two fingers.

In some variations, the method described above may be performed with an access cannula including a handle and a trocar including the finger grip. Alternatively, the method may be performed with two cannulas (once including the handle and one including the finger grip), or two trocars. The steps would be otherwise the same, as the configuration of the handle and finger grip, as well as the engagement portions and the stops may be similar. Thus, any of the methods and devices described herein may include a generic term for either or both the trocar and the access cannula (e.g., “medical instrument” or first and second interlocking elements).

### BRIEF DESCRIPTION OF THE DRAWINGS

FIGS. 1A to 1E illustrate one variation of a trocar including a handle.

FIGS. 2A to 2E illustrate the handle variation shown in FIGS. 1A to 1E including exemplary dimensions and partial cross-sectional views.

FIGS. 3A to 3H illustrate one variation of an access cannula including finger grips. The cannula may interlock with the upper handle shown in FIGS. 1A-2E.

FIGS. 4A-4E illustrate exemplary dimensions for the access cannula shown in FIGS. 3A-3H.

FIGS. 5A to 5E show one variation of a composite device including a trocar and an access cannula interlocked as described herein.

FIGS. 6A and 6B illustrate another variation of a trocar including a handle.

### DETAILED DESCRIPTION

In general, the interlocking composite devices described herein include a first element (e.g., an access cannula) having one or more finger grips spaced from the proximal end of the first element, and a second element (e.g., trocar) having a handle at its proximal end. The first and second element engage with each other and interlock so that the finger grips of the first element is offset from the handle of the second element bother laterally, and rotationally. Thus, when interlocked, the handle of the composite device does not contact the finger grips, and the handle and finger grips may be perpendicular to each other (e.g., rotated relative to each other by 90 degrees). This arrangement of finger grip and handles may allow the device to be manipulated conveniently. For example, the finger grips in the composite device may fit between a user's fingers when the user is gripping the proximal handle.

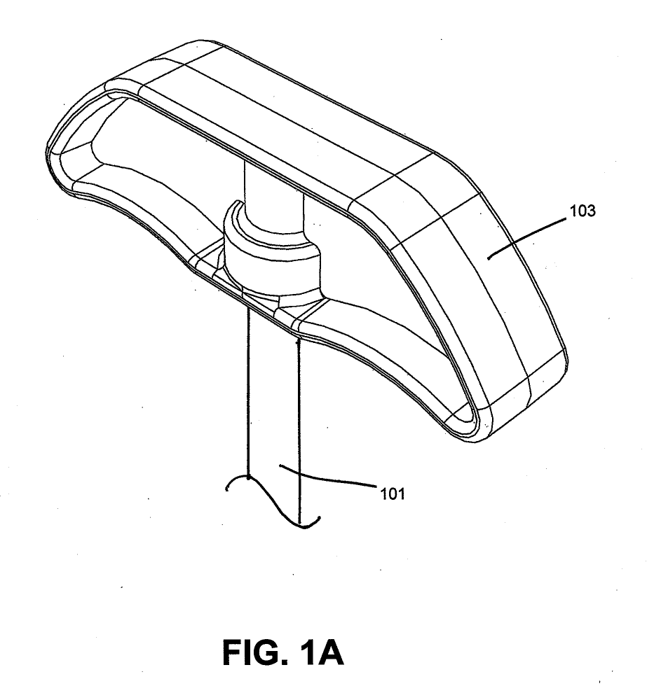

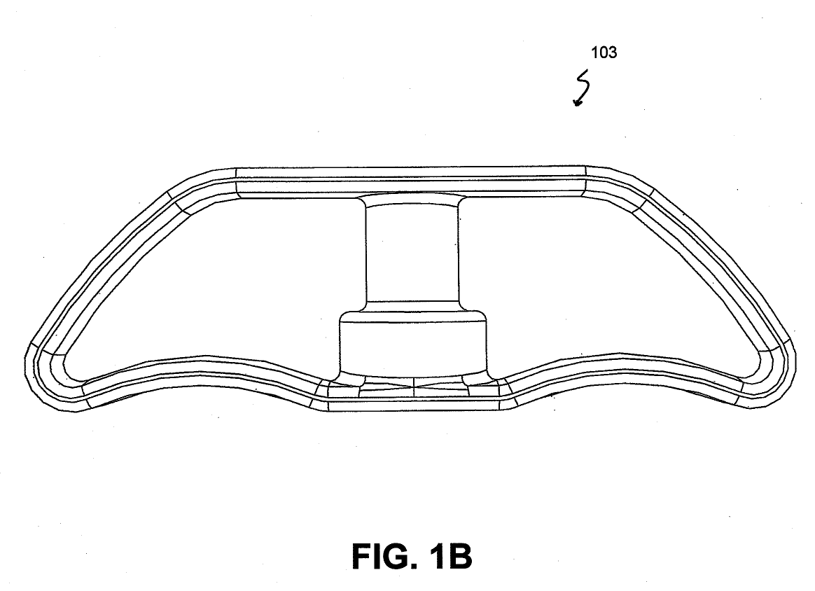

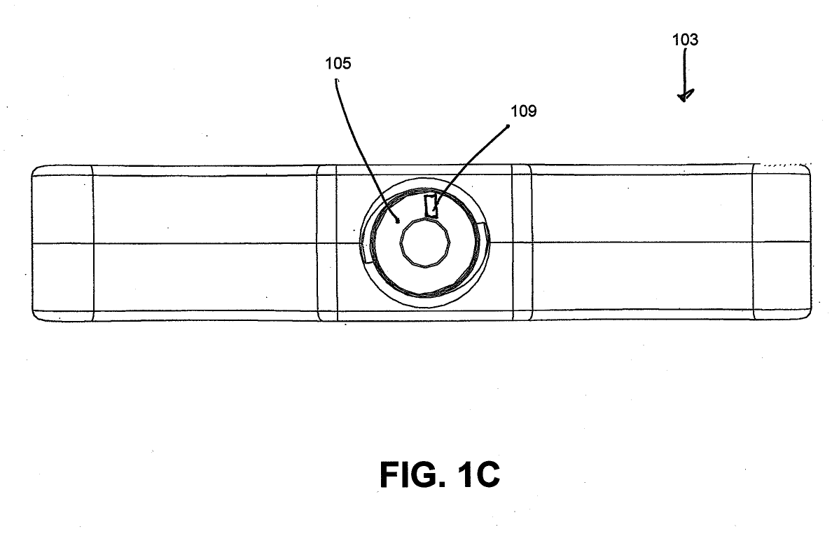

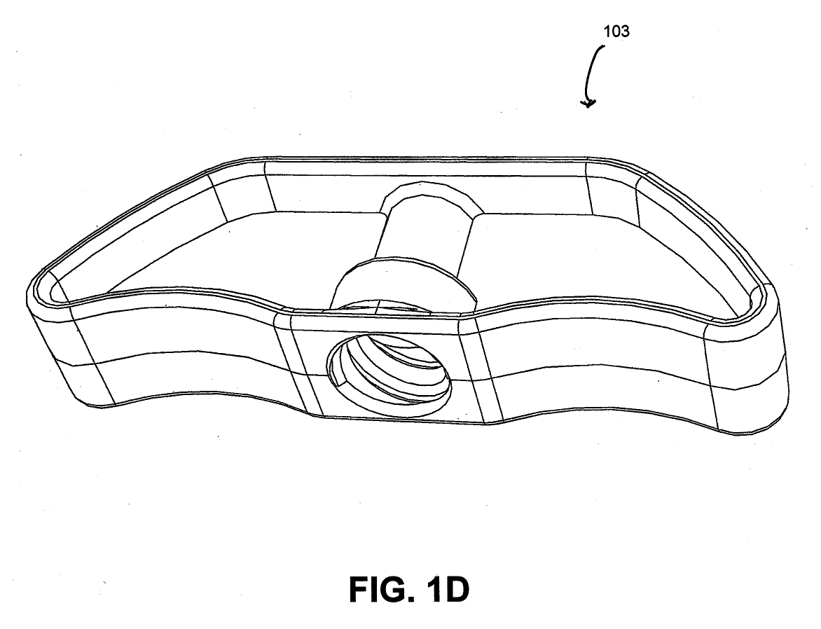

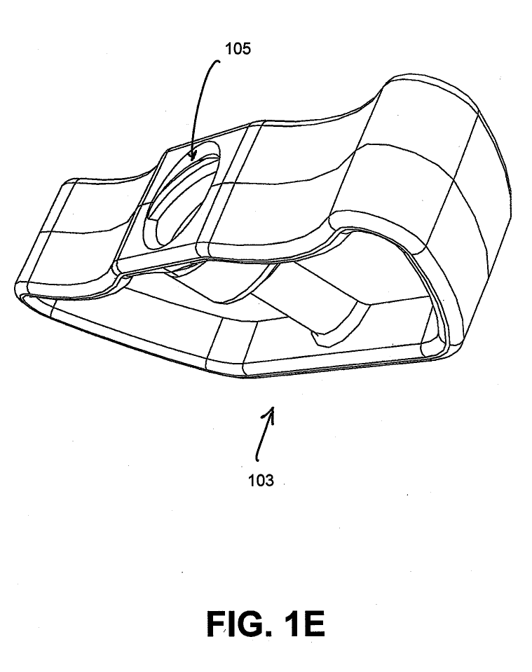

For example, FIGS. 1A to 1E illustrate one variation of a handle 103 on the proximal end of a first element. In this variation, the first element (first medical element) is a trocar 101. FIG. 1A is a side perspective view of the handle 103, showing a portion of the trocar 101. For convenience, the elongate and hollow trocar, which may include a sharp or tissue-penetrating distal end, is omitted from the rest of the figures. FIG. 1B is a side view of the handle 103, and FIG. 1C is a bottom view of the same handle. The bottom view shows the mating surface region 105 at the distal end (the bottom) of the handle. This mating surface region 105 is configured to receive an engagement region of a second element (e.g., cannula). The mating surface may be threaded so that it can mate with threads forming the engagement region of the second element. In addition, the mating surface region 105 also includes a “stop mate” that is oriented and configured to mate with a stop on the second element to maintain the rotational spacing between the handle 103 and any finger grips on the second element, as illustrated in more detail below. In this variation, the stop mate 109 is a projection having a flat surface against which the stop on a second element may abut. The stop mate 109 is positioned within the mating surface region 105 in this example, however it may be located outside of the mating surface region, or in any other position on the first element, so long as it may interact with the stop on the second element.

FIGS. 1D and 1E show additional side perspective views of the handle 103.

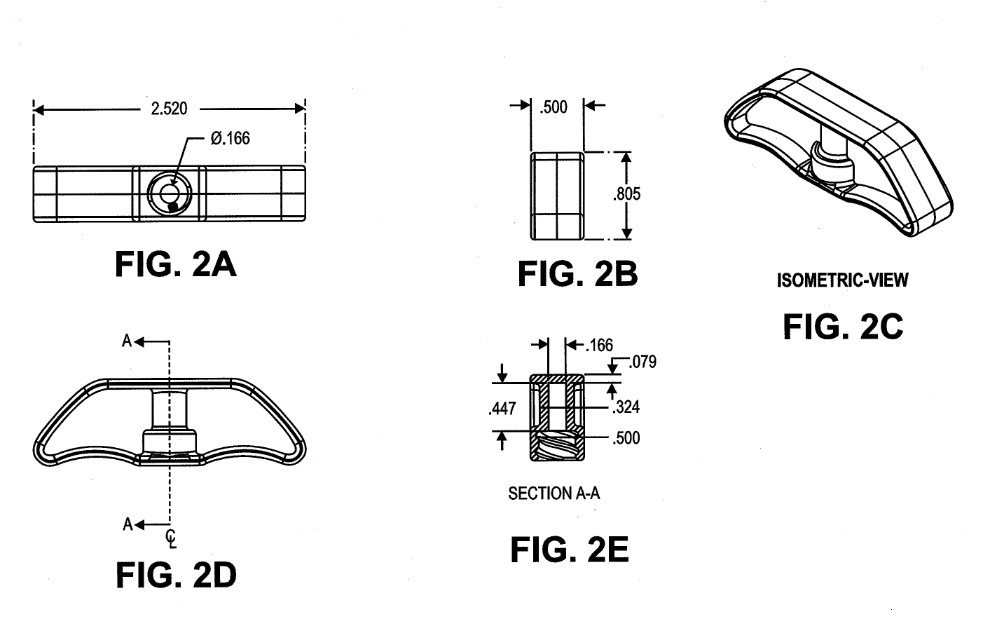

FIGS. 2A to 2E show various dimensions of the handle shown FIGS. 1A-1E. For example, the handle, which may be formed of any appropriate material, may be made approximately 2.5 inches long, and 0.5 inches thick. The handle may be fabricated (e.g., molded) using any appropriate method. In some variations, the handle is formed of a polymeric material and injection molded. FIG. 2A shows a bottom view of the handle shown in isometric view in FIG. 2C. FIGS. 2B and 2C shows a side views from two different sides, and the FIG. 2E shows a cross-sectional view through the line A-A shown in FIG. 2D. The dimensions shown are in inches, but should be understood to be approximate and exemplary. For example, the actual sizes may be expressed as +/−10% or 15% or 20% of the sizes illustrated.

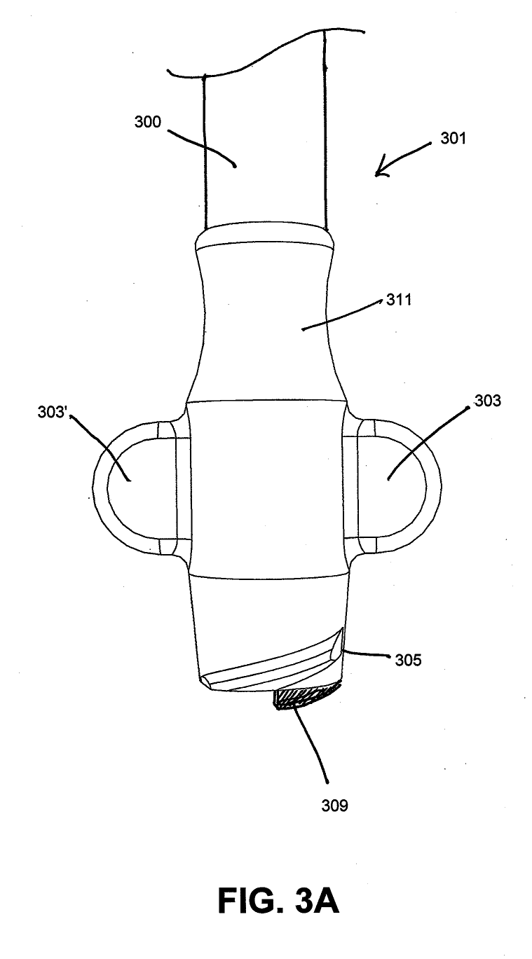

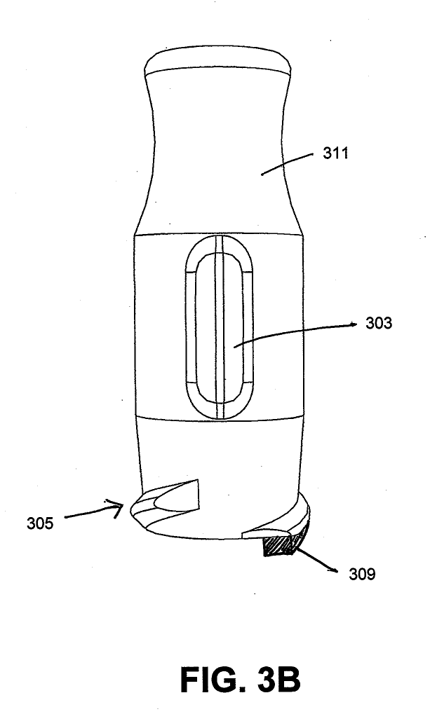

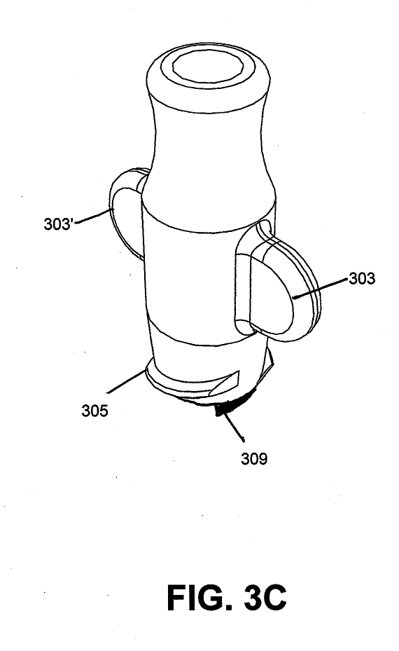

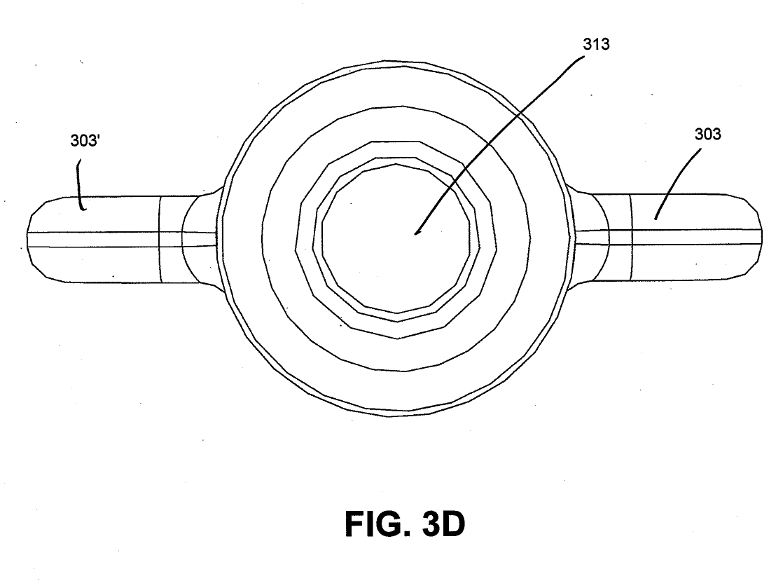

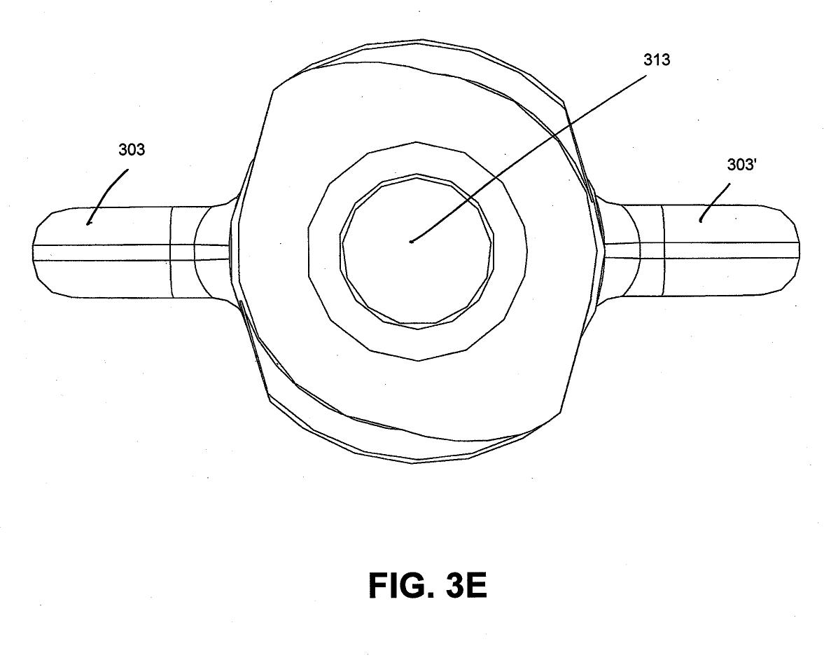

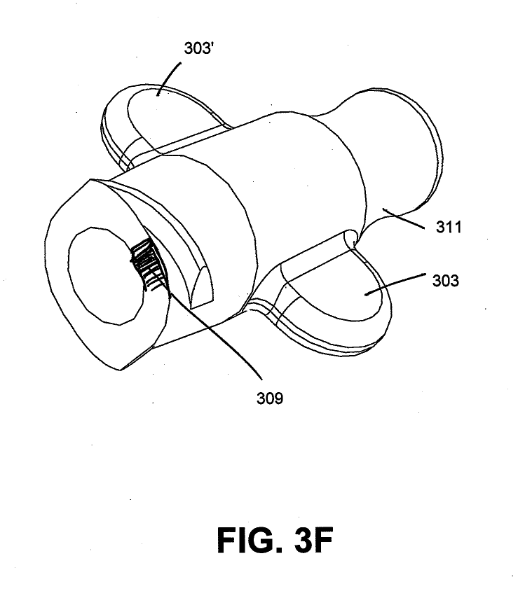

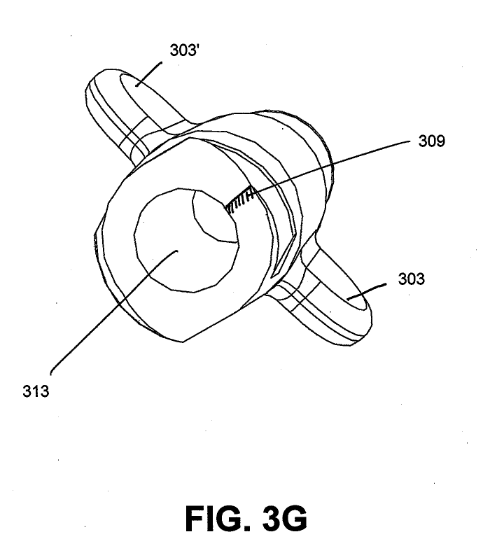

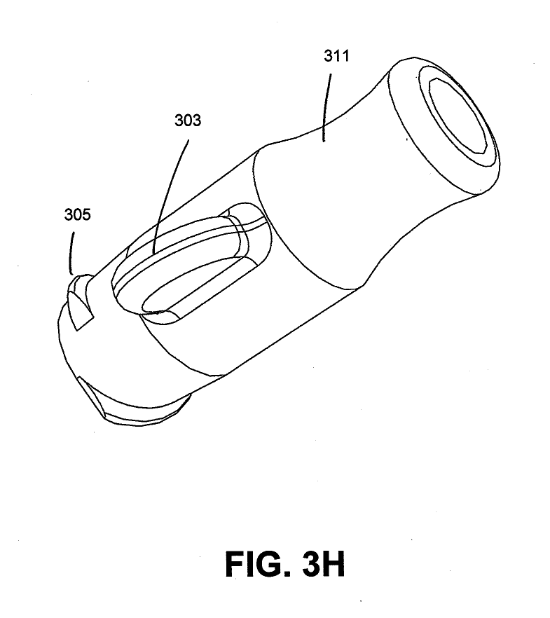

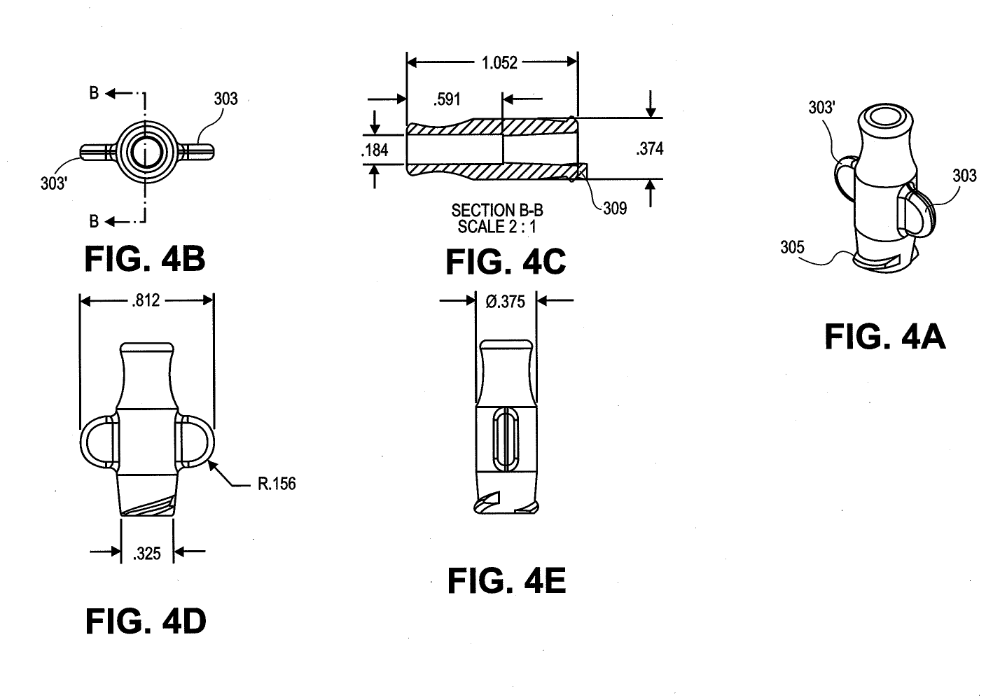

FIG. 3A shows one variation of a second element including two finger grips 303, 303′. In this example, the second element is an access cannula 301. This variation of the cannula handle 301 may mate (and interlock) with a first element such as the trocar shown in FIGS. 1A-2E. For example, FIGS. 3A and 3B show side views of the proximal end of the access cannula 301. The elongate, hollow portion forming the access cannula 300 is shown in FIG. 3A; for convenience, this portion is not illustrated in FIGS. 3B-4E. FIGS. 3B-4E all show alternative views of the proximal end of the access cannula 301. This portion of the access cannula may be referred to as the access cannula handle, since it includes the finger grip regions 303, 303′ which extend from the elongate (longitudinal) axis of the cannula. This access cannula handle portion may be secured to the proximal end of the cannula, preferably permanently, e.g., by adhesive, welding, or the like.

As illustrated in FIGS. 3A-4E, the proximate end of the cannula may include one or more finger grip regions 303, 303′. In the exemplary variation shown, a pair of finger grip regions extend from a position slightly distal to the proximal end of the cannula. The finger grip regions may be referred to as grips, tabs, wings, or tab wings. The region immediately distal to the finger grip or grips may be necked inward 311, which may both help secure the access cannula handle to the cannula, and may also provide another grasping surface.

A stop 309 is also illustrated in FIG. 3A and subsequent figures. In this example, the stop is oriented so as to engage the stop mate on the trocar (or first element) when the finger grips are offset from the handle of the first element by ninety degrees. The stop shown in FIG. 3A projects from the proximal end of the cannula, so that it may extend into a mating surface region in the handle of a first element (e.g., trocar) and mate with a stop mate. The stop is shown as a ramp that ends in a flat face; the flat face may abut a complimentary face of a stop mate.

An engagement region 305 is also shown at the proximal end of the cannula. In this variation, the engagement region 305 is configured as a threaded region that mates with a mating surface region on the distal side of a handle, as previously described and illustrated.

FIG. 3B show another side view of the proximal end of the cannula, rotated slightly from the view shown in FIG. 3A, and FIG. 3C is a side perspective view of the same. FIG. 3D is a top view, showing a central passage 313 through the cannula. As mentioned above, the first element (e.g., the trocar) may be passed through the second element (the cannula illustrated in these examples) by sliding into this central passage 313. FIG. 3E is a bottom view of just the handle portion of the cannula. The opening shown 313 may be continuous with the opening through the cannula. FIG. 3F to 3H are side perspective views.

FIGS. 4A-4E illustrates exemplary dimensions of the proximal end of the access cannula illustrated in FIGS. 3A-3H (the “access cannula handle region”). For example, FIG. 4A shows a side perspective view similar to the view shown in FIG. 3C. FIG. 4B is a top view of the same element, and FIG. 4C is a sectional view through line B-B of FIG. 4B. FIGS. 4D and 4E show side perspective views similar to the views shown in FIGS. 3A and 3B. The dimensions illustrate are only exemplary. As in FIG. 2A-2E, these dimensions are shown in inches, but the actual values provided should be understood to be approximate and exemplary. For example, the actual sizes may be +/−10% or 15% or 20% of the sizes illustrated, and may be expressed as within the range including these +/− percentages of the indicated sizes.

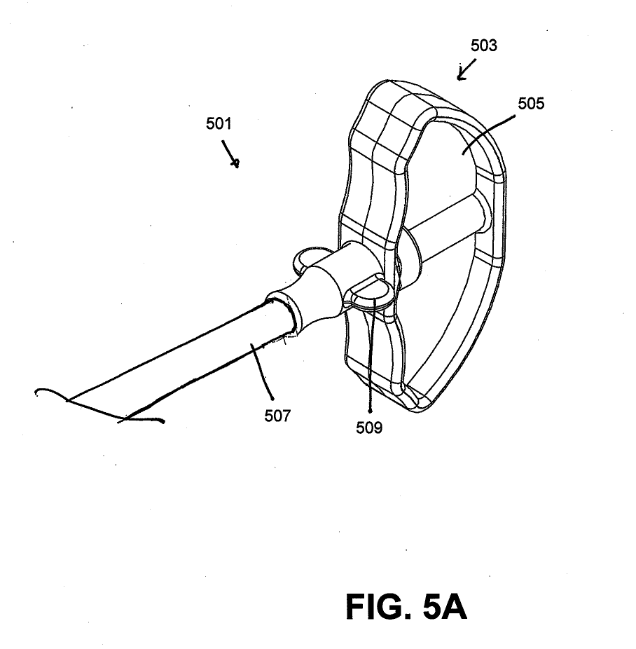

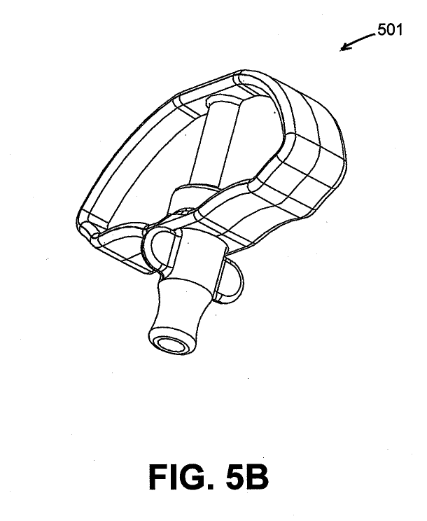

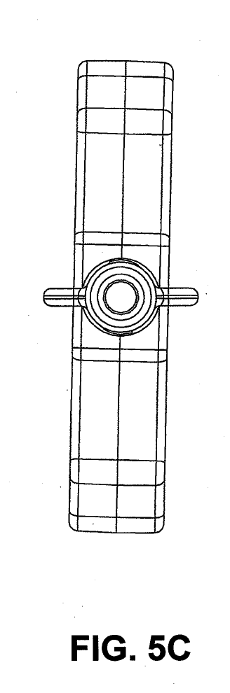

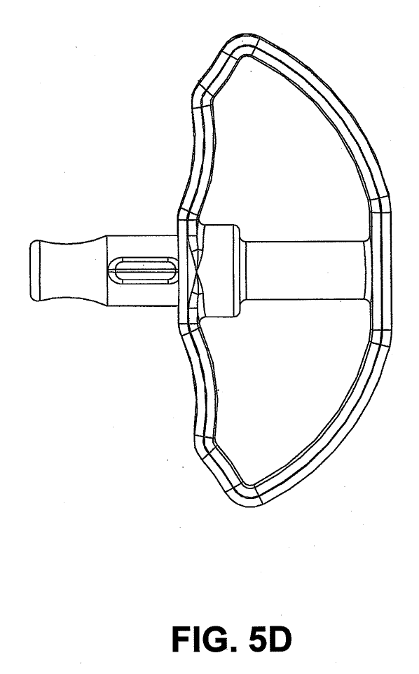

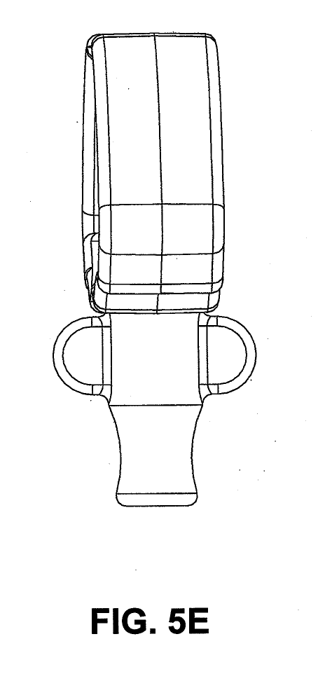

FIGS. 5A to 5E illustrate one variation of a composite device 501 formed when the first element engages with the second element. In this example, shown in FIG. 5A, the composite device includes a trocar 503 having a handle 505 that is interlocked with an access cannula 507 having a pair of finger grips 509. The finger grips 509 are offset from the handle 505 both axially and radially. For example, the finger grips do not contact the handle, and the face of the finger grips 509 are perpendicular to the face of the handle 505. In this configuration, a user operating the composite device 501 could grasp the handle while the finger grips were positioned between the user's fingers. FIG. 5B shows a perspective view of the composite device 501. FIGS. 5B to 5E all illustrate various perspective views of the composite device. For convenience, the elongated portions of the cannula and trocar have been removed from these illustrations. FIG. 5B is a side perspective view, FIG. 5C is a side view, and FIG. 5D is a bottom view of the composite device shown in FIG. 5A. FIG. 5E is another side view of this device.

In general, the first and second elements (e.g., the trocar and cannula) interlock so that the lower (cannula) handle region, including at least one finger grip, is locked in the correct orientation with respect to the upper (trocar) handle. The entire composite assembly may be manipulated, and particularly twisted or torqued, to move both the elements when the two are fully engaged. When the two are interlocked, the handle may be gripped and manipulated by itself in order to manipulate the interlocked handles once the two are engaged. In some variations, the device may include a lock to secure the first and second elements together. Alternatively, the second element (e.g., the catheter) may be manipulated individually by twisting or manipulating the finger grips while holding the handle. This sort of manipulation may be used to separate the two elements or simply to adjust the relative positions of the two.

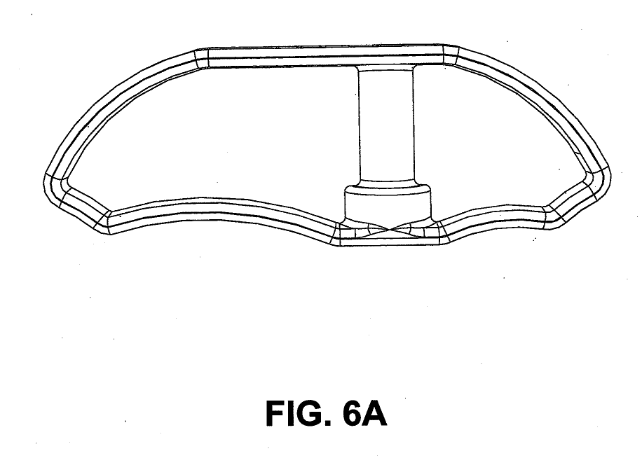

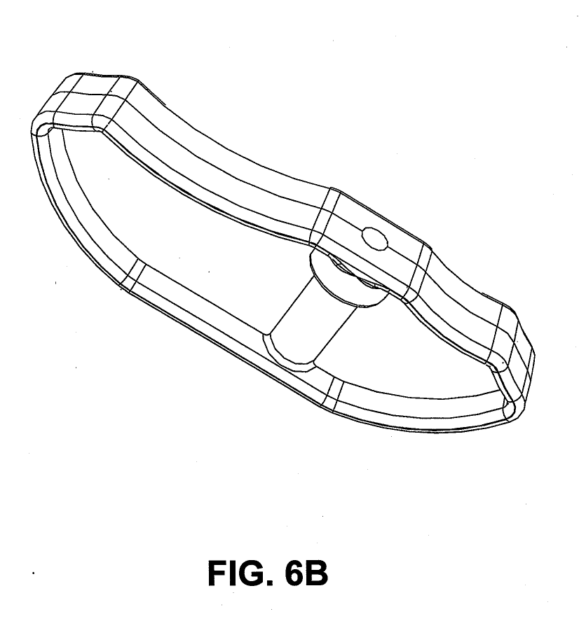

In some variations, the shape of the handle (e.g., the trocar handle) may be asymmetric in side view, as illustrated in FIGS. 6A and 6B. For example, the left side of the device shown in FIG. 6A may project further than the right side, or may trace a larger area. This asymmetry may provide a reference when rotating the device radially, and may also help in the ease of manipulation of the device by providing leverage for twisting of the device.

In operation, the interlocking composite device described herein may be operated by interlocking the first element and the second element so that the handle on the first element is offset from the finger grip on the second element both axially and radially so that the composite device may be manipulated. In one example, the second element is an access cannula having a pair of finger grip regions that extend laterally from the cannula just distally from the proximal end of the cannula, similar to the arrangement shown in FIG. 3A. The finger grip regions may be used to help grasp the cannula as it is inserted into a patient's tissue. One or more additional device (e.g., drills, distracters, etc.) may also be used to help position the cannula within a body. Thereafter a first element including a handle, such as a trocar as shown in FIG. 1A, may be engaged with the cannula element. For example, the trocar may be inserted into the cannula so that it slides within the internal passage of the cannula and the distal end of the trocar projects out of the cannula. The trocar and cannula may then be engaged with each other. For example, the trocar may be engaged with the cannula through an engagement region such as a threaded region on the cannula that interacts with a mating surface within the handle of the trocar, as illustrated in FIG. 5A. In this example, the threaded regions may interlock, securing the two elements together. When engaging the two elements such as the cannula and the trocar, the stop and the stop mate on the cannula and trocar, respectively, may ensure the orientation of the two elements. Thus, when fully engaged, the handle on the trocar and the finger grip (or grips) on the access cannula are perpendicular to each other as shown in FIG. 5A.

Thereafter, the composite device may be manipulated by holding the handle. In some variations, the handle may be held such that the finger grip regions are secured between the users fingers. This orientation may allow better leverage (better grip), and may prevent the two elements from uncoupling. Furthermore, this configuration may prevent pinching, and may allow more reliable manipulation of the composite device.

While the interlocking composite devices and methods for using them have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

We claim:

1. An interlocking trocar and access cannula tool configured to form a composite device, the tool comprising:
an access cannula comprising:
an elongate cannula body;
a finger grip surface extending from the cannula body, wherein the finger grip surface extends lateral to the cannula body from a position distal to the proximal end of the cannula body;
an engagement region at the proximal end of the cannula body; and
a stop at the proximal end region of the cannula body; and
a trocar comprising:
an elongate trocar body extending longitudinally;
a handle at the proximal end of the trocar body, the handle having a gripping surface oriented parallel to the longitudinal axis of the trocar body;
a mating surface at the distal portion of the handle configured to mate with the engagement region of the access cannula; and
a stop mate within the engagement region;
wherein the trocar is configured to slide within the elongate cannula body so that the engagement region on the access cannula mates with the mating surface on the trocar to form a composite device in which the finger grip surface of the access cannula is oriented perpendicular to the handle gripping surface of the trocar when the stop on the access cannula is mated with the stop mate on the trocar.

2. The tool of claim 1, wherein the access cannula comprises a pair of finger grips extending laterally from a portion the elongate cannula body that is distal to the proximate end.

3. The tool of claim 1, wherein the engagement region on the access cannula comprises a threaded region.

4. The tool of claim 1, wherein the stop comprises a projection extending from the proximal end of the access cannula.

5. The tool of claim 4, wherein the stop mate on the trocar comprises a face configured to abut the projection extending from the proximal end of the access cannula when the access cannula and trocar are engaged and the finger grip surface is oriented at a right angle from the handle gripping surface of the trocar.

6. The tool of claim 1, further comprising a lock configured to secure the access cannula within the trocar when the two are engaged.

7. The tool of claim 1, wherein the finger grip surface of the access cannula is configured to fit between a subject's fingers when the trocar is engaged with the access cannula to form the composite device and the subject is holding the composite device by the handle of the trocar.

8. The tool of claim 1, wherein the finger grip surface of the access cannula is configured so that it does not contact the handle of the trocar when the trocar is engaged with the access cannula to form the composite device.

9. A method of manipulating an interlocking trocar and access cannula tool configured to form a composite device, the method comprising:
inserting the distal end of a trocar into an access cannula, wherein the trocar includes a proximal handle and the access cannula includes at least on finger grip;
connecting the proximal end of the access cannula with the distal portion of the handle on the trocar to form a composite device, so that a stop on the access cannula engages a stop mate on the trocar to secure the finger grip extending from the elongate body of the access cannula oriented at a right angle to the handle of the trocar; and
manipulating the composite device while gripping the handle of the trocar.

10. The method of claim 9, wherein the step of inserting the distal end of the trocar into the access cannula comprises sliding the trocar within a channel through the access cannula.

11. The method of claim 9, wherein the step of connecting the proximal end of the access cannula with the distal portion of the handle comprises engaging an engagement region on the proximal end of the access cannula with a mating surface at the distal portion of the handle on the trocar.

12. The method of claim 9, further comprising locking the access cannula and the trocar together.

13. The method of claim 9, further comprising gripping the handle of the trocar so that the finger grip is positioned between two fingers.

14. An interlocking trocar and access cannula tool configured to form a composite device, the tool comprising:
an access cannula comprising:
an access cannula handle at the proximal end of the access cannula;
a finger grip extending from a distal portion of the access cannula handle, wherein the finger grip extends lateral to the access cannula;
a threaded engagement region at the proximal end of the access cannula handle; and
a stop extending from the proximal end region of the access cannula handle; and
a trocar comprising:
a handle at the proximal end of the trocar, the handle having a gripping surface;
a threaded receiving region within the distal portion of the handle configured to mate with the threaded engagement region of the access cannula; and
a stop mate within the engagement region configured to engage the stop on the access cannula handle;
wherein the access cannula is configured to engage with the trocar to form a composite device so that the finger grip extending from the access cannula is oriented approximately perpendicularly to the handle of the trocar when the stop on the access cannula is mated with the stop mate on the trocar.

15. The tool of claim 14, wherein the access cannula comprises a pair of finger grips extending laterally from the access cannula handle.

16. The tool of claim 14, wherein the stop mate on the trocar comprises a face configured to abut the stop extending from the proximal end of the access cannula when the trocar and access cannula are engaged and the finger grip is oriented at a right angle from the handle.

17. The tool of claim 14, further comprising a lock configured to secure the trocar within the access cannula when the two are engaged.

18. The tool of claim 14, wherein the finger grip of the access cannula is configured to fit between a subject's fingers when the trocar is engaged with the access cannula to form the composite device and the subject is holding the composite device by the handle of the trocar.

19. The tool of claim 14, wherein the finger grip of the access cannula is configured so that it does not contact the handle of the trocar when the trocar is engaged with the access cannula to form the composite device.
